(19) United States
(12) Patent Application Publication
(10) Pub. No.: US 2026/0335126 A1
(43) Pub. Date: Oct. 29, 2026

(54) THERMOREGULATORY GLABROUS SKIN WARMING DEVICE

(71) Applicant: UNIVERSITY OF MISSISSIPPI MEDICAL CENTER, Jackson, MS (US)

(72) Inventors: Jacob Blake Daniels, Jackson, MS (US); Michael Ryan Brown, Jackson, MS (US)

(21) Appl. No.: 19/658,136

(22) Filed: Apr. 24, 2026

Related U.S. Application Data
(60) Provisional application No. 63/796,239, filed on Apr. 28, 2025.

(51) Int. Cl. A61F 7/00

(52) U.S. Cl. CPC A61F 7/0085; A61F 2007/0036; A61F 2007/0054; A61F 2007/0086

(57) ABSTRACT

Disclosed is a patient warming system that targets glabrous skin regions of distal extremities to promote core body warming while preserving access to dorsal vasculature. The system includes a bladder configured to contain a heat transfer fluid, the bladder being partially contained within a moldable housing that can conform to portions of the distal extremity, such as the fingers/toes, without obstructing dorsal access. A heat exchange unit heats the heat transfer fluid, which is circulated through tubing to the bladder by a pump. The moldable housing can be manually shaped around anatomical features such as digits or joints to help maintain desired thermal contact. A method of use includes heating the heat transfer fluid, circulating it to the bladder, contacting the bladder to glabrous skin surfaces, and manually molding the housing around the patient's anatomy to maintain thermal contact between the bladder and glabrous skin surfaces.

### CROSS-REFERENCE TO RELATED APPLICATIONS

This application claims priority to and the benefit of United States Provisional Application No. 63/796,239, filed on Apr. 28, 2025, the entirety of which is incorporated herein by reference.

### BACKGROUND

### Technical Field

The present disclosure relates to patient warming systems, and more particularly to devices and systems for warming glabrous skin surfaces of distal extremities to maintain or elevate a patient's core body temperature, such as during surgical procedures, dialysis, recovery, or patient transport where access to dorsal vasculature is desired.

### BACKGROUND

Maintaining core body temperature is critical during surgical and medical procedures to prevent hypothermia and its associated complications. Patients under anesthesia or undergoing procedures involving large fluid exchanges, such as burn debridement, dialysis, or childbirth, are especially vulnerable to drops in core body temperature. Hypothermia has been associated with increased risk of infection, increased blood loss, prolonged recovery time, and reduced overall thermal stability.

Accordingly, there remains a need for patient warming systems that can address these problems.

### SUMMARY

The present disclosure provides systems and methods for warming a patient by targeting glabrous skin regions of distal extremities, such as the palm of the hand or sole of the foot. A patient warming system according to various embodiments includes a warming assembly comprising a bladder configured to receive a heat transfer fluid and a moldable housing at least partially surrounding the bladder. The moldable housing is configured to conform to the distal extremity while leaving dorsal vasculature exposed to maintain access for intravenous therapy or other procedures. In some embodiments, the moldable housing includes a support material having a rigid, plastically deformable core and a cushioning layer, allowing manual shaping around anatomical features such as digits, a wrist, or an ankle. The warming assembly may further include an attachment element, such as a strap, or may be secured by the moldable housing without a strap.

The system further includes a heat exchange unit comprising a reservoir configured to contain the heat transfer fluid and a heating element configured to heat the heat transfer fluid, and a pump configured to circulate the heat transfer fluid. The heating element may be disposed within the reservoir or external to the reservoir and configured to transfer heat to the heat transfer fluid. In some embodiments, the heat exchange unit is configured to maintain the heat transfer fluid within a target temperature range, such as approximately 95°F to 120°F. The system may further include a temperature sensor configured to measure a temperature of the heat transfer fluid and a controller configured to regulate operation of the heating element based on the measured temperature, including deactivation of the heating element when a threshold temperature is exceeded.

A fluid distribution system is fluidically coupled to the heat exchange unit and is configured to deliver heated heat transfer fluid to one or more warming assemblies and return cooled fluid to the heat exchange unit. The fluid distribution system may include at least one supply flow path and at least one return flow path, and in some embodiments includes a manifold having an inlet and a plurality of outlet ports. The manifold may be configured to distribute heated fluid to a plurality of warming assemblies, enabling simultaneous warming of multiple distal extremities.

In some embodiments, the fluid distribution system includes one or more connectors configured to selectively couple and decouple portions of the system. The connectors may include quick-disconnect couplings configured to restrict fluid flow when disconnected and may be positioned to separate sterile and non-sterile portions of the system. Such configurations can facilitate clinical workflows by allowing disposable patient-contact components to be separated from reusable system components.

The system may further include a portable enclosure housing at least the heat exchange unit and the pump, the enclosure being configured for placement adjacent to a patient support structure, such as a hospital bed. The system may be configured to operate with a plurality of warming assemblies, each fluidically coupled via respective supply and return flow paths, allowing modular and scalable warming configurations.

Methods of use include heating the heat transfer fluid within the heat exchange unit, circulating the heat transfer fluid using the pump, distributing the heat transfer fluid to one or more warming assemblies, contacting glabrous skin of one or more distal extremities with the bladder, and transferring heat from the heat transfer fluid to the patient. In some embodiments, the method further includes manually molding the moldable housing around the distal extremity and optionally securing the warming assembly using an attachment element.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates a top view of a patient warming system, including the heat exchange unit, pump, tubing, and bladder assembly.

FIG. 2 illustrates a schematic view showing the heat exchange unit, the pump, and two pairs of tubing segments configured to deliver heated fluid to and return cooled fluid from multiple bladders.

FIGS. 3A and 3B illustrate a patient’s hand positioned on the warming device, with a strap securing the moldable housing around the hand.

FIGS. 4A and 4B illustrate a perspective view of a patient’s foot positioned on the warming device.

FIG. 5 illustrates the warming device after molding around a patient’s hand, with a portion of the central bladder exposed to enable direct contact with the glabrous skin.

FIG. 6 shows resulting impressions after removal of the hand from a warming device.

FIG. 7 illustrates the interior of the warming device showing a supply tube entering the bladder, forming a looped/serpentine flow path within the bladder, and an outlet tube exiting the bladder carrying cooled fluid.

FIG. 8 illustrates a perspective view of a patient warming system including a portable enclosure housing a heat exchange unit and associated components, the enclosure being configured for placement adjacent to a patient support structure.

FIG. 9 illustrates a schematic view of a fluid distribution system including a supply-side manifold and a return-side manifold configured to distribute heat transfer fluid to and from a plurality of warming assemblies.

FIG. 10 illustrates an internal view of the portable enclosure showing an arrangement of system components including a reservoir, insulation, power supply, controller, and associated fluid and electrical connections.

### DETAILED DESCRIPTION

### Introduction

The following detailed description sets forth various embodiments of patient warming systems and methods of use. Although specific embodiments are described herein, it will be understood that the invention may be embodied in many different forms and should not be construed as limited to the particular structures or methods set forth. Rather, the embodiments described are provided so that this disclosure will be thorough and complete, and will fully convey the scope of the invention to those skilled in the art.

Various conventional warming systems, including forced-air warming devices, conductive pads, and fluid-based heating systems, have been developed to stabilize body temperature during medical interventions.

Many existing patient warming devices utilize large blankets, wraps, or mitt-style systems that cover significant portions of a patient's body, including the hands and feet. While such systems can deliver heat through convection, conduction, or forced-air mechanisms, their size and bulk can restrict access to the patient, particularly to dorsal veins of the hands and feet that are commonly used for catheter placement during intravenous therapy, dialysis, and other procedures. In addition, some forced-air systems may disrupt sterile airflow within clinical environments, potentially increasing infection risk.

Other warming approaches, including fluid-circulating pads and localized heating devices, may improve heat transfer efficiency but can present additional limitations. For example, certain systems lack the ability to simultaneously warm multiple distal extremities in a controlled and modular manner, limiting overall heat transfer efficiency. In addition, some systems do not adequately separate sterile and non-sterile components, complicating use in surgical settings. Other devices may lack robust temperature monitoring and control, increasing the risk of overheating or inconsistent thermal delivery. Still further, existing systems may be limited in portability, requiring fixed installations that do not readily support patient transport or bedside mobility.

Furthermore, some devices rely on complex mechanisms such as negative pressure systems to enhance heat transfer, which may increase system complexity and interfere with clinical access or ease of use. Other systems may include fluid or electrical components that are not optimally configured for safety, such as arrangements that increase risk of fluid leakage, air entrapment, or unintended exposure to heated elements.

Accordingly, there remains a need for improved patient thermoregulatory systems that provide efficient heat transfer to targeted anatomical regions, such as glabrous skin surfaces, while maintaining access to dorsal vasculature, supporting sterile clinical workflows, enabling controlled and safe temperature regulation, and allowing for modular, portable, and scalable operation across one or more distal extremities.

The systems described herein address the limitations of conventional approaches by providing heat transfer to glabrous skin of a patient. Glabrous skin, found on the palms of the hands and soles of the feet, is highly specialized for thermal exchange due to its dense concentration of arteriovenous anastomoses and rich vascular networks. These anatomical features allow for rapid and efficient heat transfer between the skin and the core body circulation. By directing warming efforts toward glabrous skin surfaces, a patient warming system can achieve effective elevation of core body temperature while minimizing the need to cover large areas of the body. This targeted approach provides effective thermal efficiency, preserves access to dorsal venous structures, and reduces the risk of excessive heating of non-target tissues.

In various embodiments, the system is configured to circulate a heat transfer fluid in a closed-loop configuration. The system can include a heat exchange unit comprising a reservoir configured to contain the heat transfer fluid and a heating element configured to heat the heat transfer fluid. The heating element may be disposed within the reservoir or external to the reservoir (e.g., configured to transfer heat through a wall of the reservoir). In some embodiments, the reservoir may be insulated to reduce thermal losses and improve heating efficiency.

The system can further include a pump configured to circulate the heat transfer fluid through a fluid distribution system. The fluid distribution system may include one or more supply flow paths configured to deliver heated fluid and one or more return flow paths configured to return cooled fluid to the heat exchange unit, thereby forming a continuous circulation loop. In certain embodiments, the system includes a manifold having an inlet and a plurality of outlet ports configured to distribute fluid to multiple warming assemblies. The manifold may be configured to support multiple simultaneous connections, such as two, three, or four outlet ports, enabling scalable warming of multiple distal extremities.

In some embodiments, the fluid distribution system includes connectors positioned along one or more flow paths. The connectors may be configured to selectively couple and decouple portions of the system, and may include quick-disconnect couplings configured to automatically restrict fluid flow when disconnected. In certain configurations, the connectors are positioned to separate sterile and non-sterile portions of the system, allowing reusable components, such as the heat exchange unit and pump, to remain outside a sterile field while disposable and/or patient-contact components are used within the sterile field.

Each warming assembly may include a bladder configured to receive the heat transfer fluid and to contact a glabrous skin surface of a distal extremity. The bladder may be formed from a flexible, fluid-compatible material and may include internal flow paths, such as serpentine or coiled tubing arrangements, to promote uniform heat distribution across a contact surface. The bladder may be partially surrounded by a moldable housing configured to conform to the distal extremity while leaving dorsal vasculature exposed.

The moldable housing may include a support material having a rigid, plastically deformable core, such as an aluminum or similar metal sheet, combined with a cushioning layer, such as a foam material. This construction allows the housing to be manually shaped at ambient conditions to conform to anatomical features such as digits, joints, or surrounding structures, and to retain its shape after molding. In some embodiments, the housing may be configured to secure the warming assembly without additional fasteners, while in other embodiments an attachment element, such as a strap, may be provided to secure the warming assembly across a dorsal side of the distal extremity.

In certain embodiments, the system includes a plurality of warming assemblies, each fluidically coupled to the fluid distribution system. The plurality of warming assemblies may be used to simultaneously warm multiple distal extremities, such as both hands, both feet, or a combination of hands and feet, thereby increasing overall heat transfer efficiency.

The system may further include control and safety features configured to regulate operation and improve reliability. For example, a temperature sensor, such as a thermocouple, may be positioned within the system to measure the temperature of the heat transfer fluid, including at or near an outlet of the heat exchange unit. A controller may be configured to regulate operation of the heating element based on the measured temperature, including automatically deactivating the heating element when a threshold temperature is exceeded. Such configurations may reduce the risk of overheating and improve consistency of delivered thermal energy. In certain embodiments, the enclosure forms an integrated system housing configured to contain and organize heating, fluid circulation, and control components.

In some embodiments, the system is housed within a portable enclosure configured to contain at least the heat exchange unit, pump, and associated components. The enclosure may include structural features to support placement adjacent to a patient, such as on a table or attached to a patient support structure, and may include internal separation between fluid-containing components and electrical components to improve safety. The enclosure may further include access features such as a fill port for the reservoir and a cover for protecting fluid distribution components such as manifolds and connectors.

A method of using the system may include heating the heat transfer fluid within the heat exchange unit, circulating the heat transfer fluid using the pump, distributing the heat transfer fluid through the fluid distribution system to one or more warming assemblies, contacting glabrous skin of one or more distal extremities with the warming assemblies, and transferring heat from the heat transfer fluid to the patient. In some embodiments, the method further includes manually molding the moldable housing around the distal extremity and optionally securing the warming assembly using an attachment element.

The systems and methods disclosed herein provide advantages over traditional patient warming devices, including high thermal efficiency through targeted warming of glabrous skin, preservation of access to dorsal vasculature, a modular and scalable configuration for multiple extremities, improved sterile field management through separable components, enhanced safety through temperature monitoring and control, and improved portability and usability in clinical environments.

### Example Embodiments

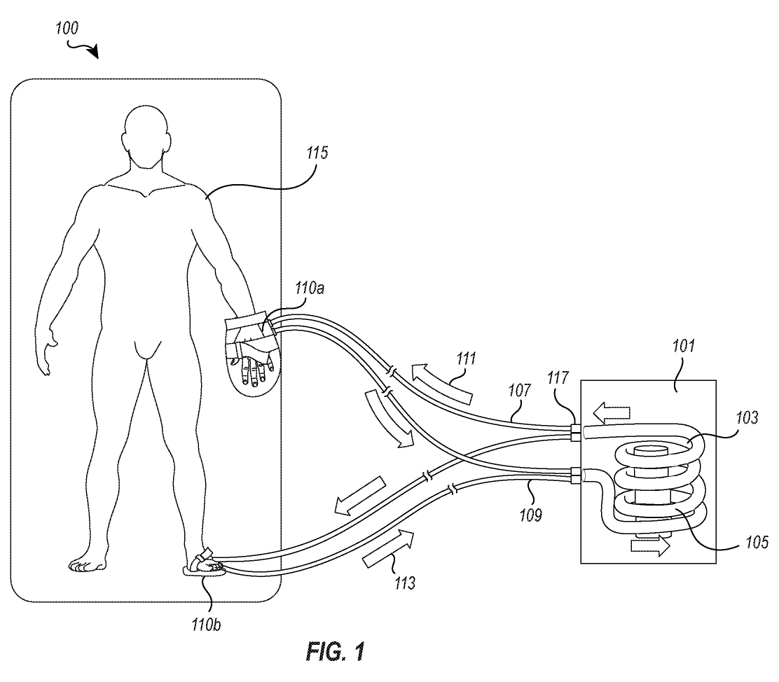

Referring now to FIG. 1, a patient warming system 100 is shown in use with a patient 115 lying down. The system 100 includes a heat exchange unit 101 configured to heat a heat transfer fluid. The heat exchange unit 101 includes a fluid reservoir 105 and, in the illustrated embodiment, a coil-shaped conduit 103 associated with the reservoir for transferring heat to the heat transfer fluid. The heat exchange unit 101 may include a heating element disposed within the reservoir or otherwise thermally coupled to the reservoir, and in some embodiments may include insulation or other thermal management features to improve heating efficiency and reduce heat loss. The heat exchange unit 101 may further include a temperature sensor and a controller configured to regulate operation of the heating element, including automatic deactivation when a threshold temperature is exceeded.

Heated heat transfer fluid exits the heat exchange unit 101 through a heated tube 107. The heated tube 107 is coupled to a connector 117, which provides fluid communication between the heat exchange unit 101 and one or more distal extremity warming assemblies. In some embodiments, the connector 117 comprises a detachable or quick-disconnect coupling configured to selectively couple and decouple portions of the system and to restrict fluid flow when disconnected. The connector 117 may further be positioned to define a boundary between a non-sterile portion of the system, including the heat exchange unit 101 and associated components, and a sterile portion of the system including tubing and patient-contact components, thereby facilitating use in clinical environments. In some embodiments, connectors may include keyed or gendered configurations configured to prevent incorrect coupling of supply and return flow paths. Connectors may include internal valves, seals, or automatic shutoff mechanisms configured to prevent fluid leakage upon disconnection.

A first bladder 110a is configured to conform to the hand of the patient 115, while a second bladder 110b is configured to conform to the foot of the patient 115. Each bladder 110a, 110b is configured to contact glabrous skin of a respective distal extremity while leaving dorsal vasculature exposed to maintain access for clinical procedures. In some embodiments, the bladders may be formed from flexible, fluid-compatible materials and may include internal flow paths configured to distribute the heat transfer fluid across a contact surface.

Heated fluid flows in direction 111 through the heated tube 107 and is directed toward the bladders 110a, 110b. In the illustrated embodiment, the heated tube 107 may branch to supply multiple warming assemblies, and in some embodiments the system may include a fluid distribution system configured to distribute heated fluid to a plurality of warming assemblies for simultaneous warming of multiple distal extremities. After heat transfer to the patient's glabrous skin, cooled fluid exits the bladder assemblies via return flow paths 113. The cooled fluid is collected through return tubes 109 and routed back toward the heat exchange unit 101 for reheating and recirculation.

The system thus establishes a continuous closed-loop flow, wherein heat transfer fluid is circulated from the reservoir 105, heated, delivered to one or more warming assemblies, and returned via the return flow tubes 109 to maintain the patient's core body temperature. Continuous circulation of the heat transfer fluid can improve thermal transfer efficiency and reduce temperature gradients at the skin interface. In some embodiments, components that contact the patient, such as the bladders and associated tubing, may be disposable, while the heat exchange unit and pump are reusable components positioned outside the sterile field.

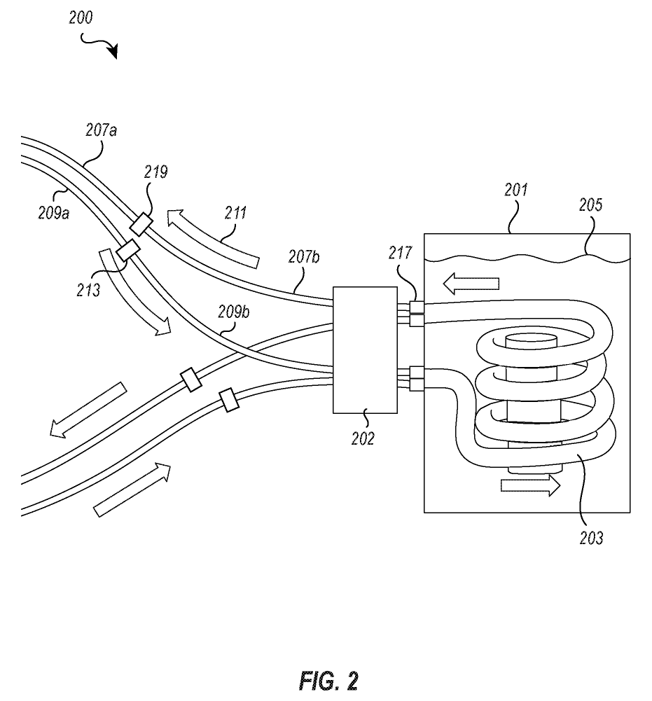

FIG. 2 shows a schematic view of an embodiment of a patient warming system 200. The patient warming system 200 can include features described in relation to any other embodiment disclosed herein, including those described in connection with FIG. 1. In this embodiment, the system includes a heat exchange unit 201 configured to heat a heat transfer fluid, such as water, saline, or a glycol solution. A fluid reservoir 205 is contained within the heat exchange unit 201 and is configured to hold the heat transfer fluid. A heating element 203 disposed within the reservoir 205 facilitates heating of the fluid. In other embodiments, the heating element may be external to the reservoir and configured to transfer heat through a wall of the reservoir. The heat exchange unit 201 may further include thermal management features, such as insulation, to improve heating efficiency and reduce heat loss.

Heated fluid exits the heat exchange unit 201 via a connector 217, which couples the heat exchange unit 201 to a supply tube segment 207b configured to carry heated fluid away from the heat exchange unit in direction 211. The system 200 may further include a temperature sensor, such as a thermocouple, configured to measure the temperature of the heat transfer fluid, for example at or near an outlet of the heat exchange unit 201, and a controller configured to regulate operation of the heating element based on the measured temperature, including automatic shutoff when a threshold temperature is exceeded.

In use, the heat exchange unit 201 and supply tube segment 207b are positioned outside of a sterile field of a procedure. The non-sterile supply segment 207b is coupled to a connector 219, which provides a transition to a sterile supply segment 207a that can be routed into the sterile field and connected to one or more warming assemblies (not shown in this expanded view). Connector 219 may comprise a quick-disconnect coupling configured to selectively couple and decouple the sterile and non-sterile segments and to automatically restrict or stop fluid flow when disconnected. In some embodiments, the connector may be keyed or otherwise configured to reduce the likelihood of incorrect coupling.

After delivering heated fluid to the warming assemblies, cooled fluid is returned through a sterile return tube segment 209a, which is coupled via a connector 213 to a non-sterile return segment 209b. Connector 213 may similarly comprise a quick-disconnect coupling configured to separate sterile and non-sterile portions of the return flow path and to prevent fluid leakage upon disconnection. The configuration shown in FIG. 2 provides a controlled interface between sterile and non-sterile portions of the system, allowing reusable components, such as the heat exchange unit 201 and pump 202, to remain outside the sterile field while patient-contact components are disposed within the sterile field. In some embodiments, the sterile tubing segments and associated warming assemblies may be disposable components.

Although a single supply and return path is illustrated for clarity, it will be understood that the system may include a plurality of such supply and return paths. In certain embodiments, a fluid distribution system including a manifold may be provided to distribute heated fluid from the heat exchange unit 201 to multiple warming assemblies and to collect return flow from the assemblies, thereby enabling simultaneous warming of multiple distal extremities in a scalable and modular configuration.

The illustrated system 200 further includes a pump 202 configured to circulate the heat transfer fluid throughout the system. For example, the pump 202 can draw cooled fluid through the return tubing 209b and direct it back into the reservoir 205 for reheating, thereby establishing a continuous closed-loop circulation system. The pump 202 may additionally or alternatively be configured to circulate heated fluid through the supply tubing. The pump 202 is configured to provide sufficient fluid pressure to drive movement of the heat transfer fluid through the system, and any suitable pump system may be utilized. In some embodiments, the pump comprises a peristaltic pump that engages the tubing externally, which can reduce contamination risk and simplify sterilization. In certain configurations, the pump may be positioned relative to the reservoir to facilitate priming and reduce air entrainment within the system. Continuous circulation of the heat transfer fluid through the system can improve thermal transfer efficiency by maintaining consistent fluid temperature and reducing thermal gradients. In some embodiments, flow paths may be visually distinguishable, such as by color coding of supply and return tubing, to facilitate correct setup and reduce the likelihood of connection errors in clinical environments.

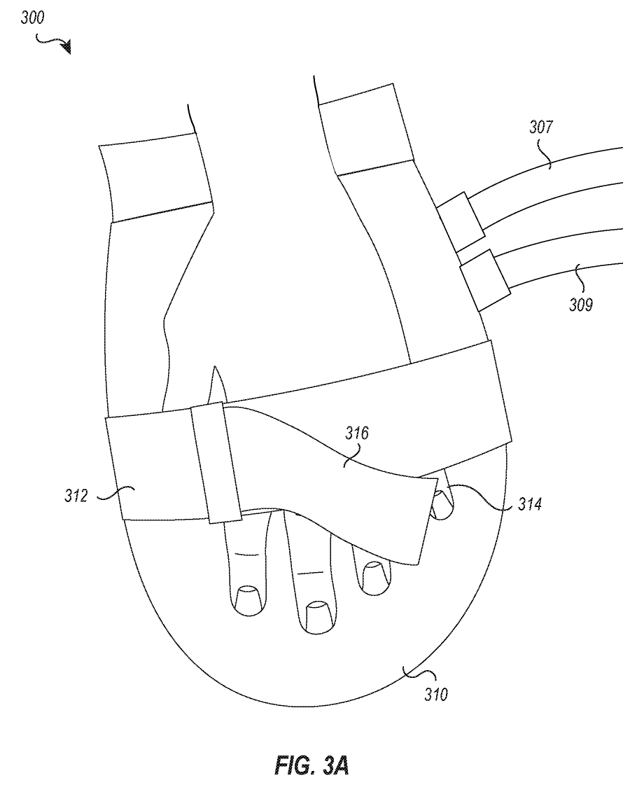

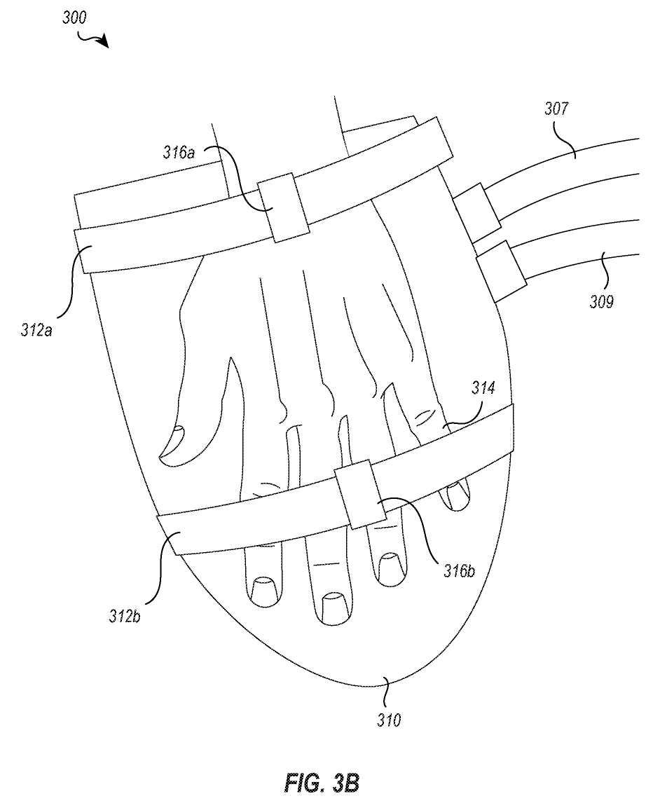

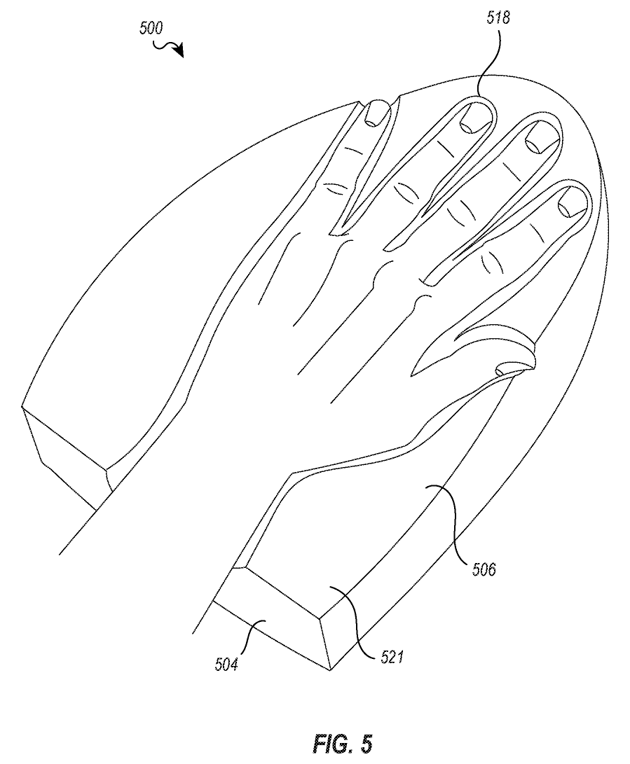

Referring now to FIGS. 3A and 3B, perspective views of a patient warming assembly positioned on a patient’s hand are shown. The patient warming system 300 can include features described in relation to any other embodiment disclosed herein. A bladder 310 configured to contain and circulate a heat transfer fluid is partially contained within a moldable housing (described in more detail below; see FIG. 5). In use, the patient’s hand 314 is positioned such that the glabrous skin of the palm is in thermal contact with the bladder 310, facilitating efficient heat transfer between the circulating fluid and the patient. The bladder 310 may include internal flow paths configured to distribute the heat transfer fluid across a contact surface to promote uniform heating. One or more supply and return tube segments 307, 309 are fluidically coupled to the bladder 310 to deliver heated fluid and return cooled fluid, respectively. In some embodiments, these tubing segments may be flexible and configured for routing within a clinical environment, and may be visually distinguishable, such as by color coding, to differentiate supply and return flow paths and reduce the likelihood of connection errors.

A fastening mechanism such as strap 312 can be included to secure the warming assembly relative to the patient’s hand. As shown, the strap 312 extends across the dorsal side of the hand, positioning the bladder 310 against the glabrous surface while leaving dorsal vasculature exposed for intravenous access or other clinical procedures. The strap 312 may be attached to the bladder 310 and/or the moldable housing at locations selected to minimize interference with clinical access points. The strap 312 can be secured using a fastener 316, which may include, for example, a mechanical clasp, snap, friction buckle, hook-and-loop fastener, or other adjustable fastening mechanism, allowing for rapid attachment and removal in clinical settings. In some embodiments, the warming assembly may include a disposable or removable barrier layer disposed over at least a portion of the bladder 310, such as a fluid-resistant sleeve or covering, to provide a sterile interface between the patient and reusable system components. This configuration can facilitate infection control and enable reuse of non-patient-contact components while maintaining a sterile patient-contact surface.

FIG. 3B shows a configuration in which the strap 312 is relatively thinner than the configuration of FIG. 3A. Thinner straps can provide sufficient retention of the bladder 310 against the glabrous skin while further minimizing interference with dorsal surfaces and maintaining improved access to clinical sites. The strap 312 may have a width between approximately 0.5 inches and 1 inch, such as between about 0.6 inches and 0.9 inches or between about 0.7 inches and 0.8 inches, or may have a width within a range defined by any two of the foregoing values. In some embodiments, the strap geometry and placement may be selected to balance securement, patient comfort, and unobstructed access to dorsal vasculature. In certain embodiments, straps may be omitted entirely. For example, the moldable housing may be manually shaped to conform to anatomical features of the hand, such as digits and joints, to retain the warming assembly in place without the need for additional fastening mechanisms. This strapless configuration can reduce setup time, improve patient comfort, and further minimize obstruction of dorsal surfaces. Additionally, the warming assembly may be configured as part of a modular system in which multiple assemblies are independently attachable to different extremities, enabling simultaneous warming of multiple sites while maintaining flexibility in clinical use.

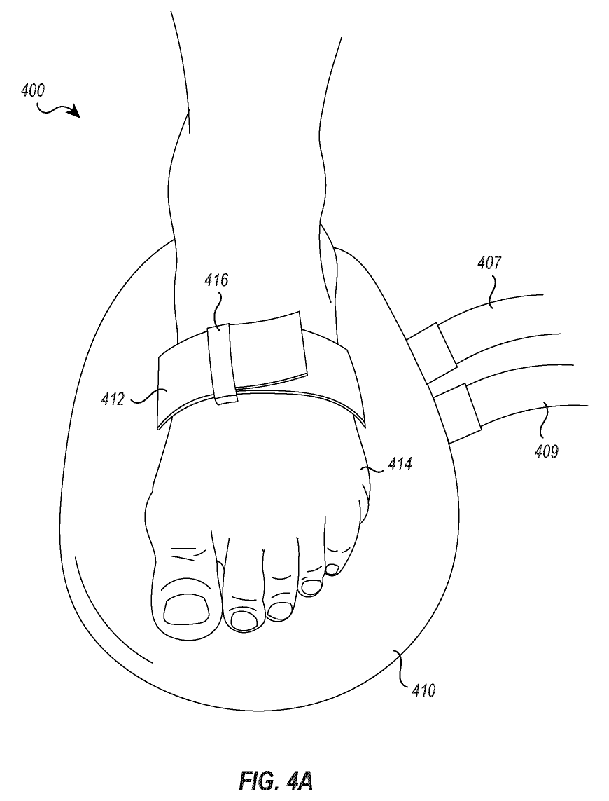

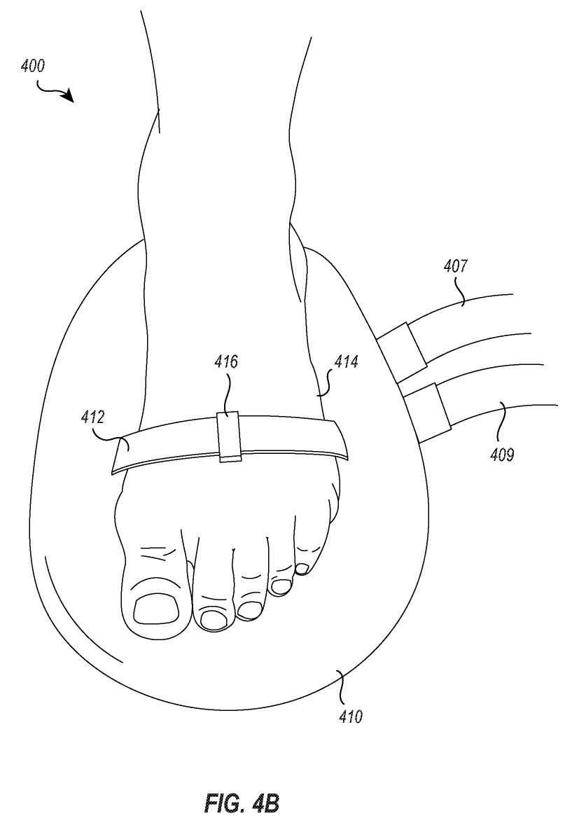

FIGS. 4A and 4B illustrate perspective views of a patient warming assembly positioned on a patient’s foot. The patient warming system 400 can include features described in relation to any other embodiment disclosed herein. In some embodiments, the same warming assembly may be configured for use on one or both hands and/or one or both feet, providing a universal or interchangeable design across different distal extremities. The geometry of the bladder and moldable housing may be configured to accommodate both palmar and plantar surfaces, enabling a common device design to be applied across multiple anatomical locations. This interchangeability can reduce system complexity and improve ease of use by allowing a single type of warming assembly to be used for multiple extremities. A bladder 410 configured to contain and circulate a heat transfer fluid is partially contained within a moldable housing. The patient’s foot 414 is positioned such that the glabrous skin of the sole is in thermal contact with the bladder 410 to facilitate efficient heat transfer.

One or more supply and return tube segments 407, 409 are fluidically coupled to the bladder 410 to deliver heated fluid and return cooled fluid, respectively. In some embodiments, the tubing may be flexible and configured for routing within a clinical environment, and may be visually distinguishable, such as by color coding, to differentiate supply and return flow paths and reduce the likelihood of connection errors. The bladder 410 may include internal flow paths configured to distribute the heat transfer fluid across a contact surface to promote uniform heating of the glabrous skin.

A fastening mechanism such as strap 412 can be positioned to extend across the dorsal side of the foot. The strap 412 can be attached or anchored to the bladder 410 and/or the moldable housing such that it is positioned over a metatarsal region (see, e.g., FIG. 4B), thereby retaining the bladder 410 and moldable housing relative to the foot while leaving dorsal vasculature accessible for clinical procedures. The strap 412 can be secured using a fastener 416, which may include, for example, a mechanical clasp, snap, friction buckle, hook-and-loop fastener, or other adjustable fastening mechanism, allowing for rapid attachment and removal in clinical settings.

In some embodiments, the warming assembly may include a disposable or removable barrier layer disposed over at least a portion of the bladder 410, such as a fluid-resistant sleeve, to provide a sterile interface between the patient and reusable components of the system. This configuration can facilitate infection control and enable separation of sterile and non-sterile components during use.

As discussed in reference to FIGS. 3A and 3B, the strap 412 may have a width between approximately 0.5 inches and 1 inch, such as between about 0.6 inches and 0.9 inches or between about 0.7 inches and 0.8 inches, or may have a width within a range defined by any two of the foregoing values. The strap geometry and placement may be selected to provide sufficient retention of the bladder 410 against the glabrous skin while minimizing interference with dorsal surfaces and maintaining access to clinical sites.

In some embodiments, straps may be omitted. For example, the moldable housing may be manually shaped to conform to anatomical features of the foot, such as the arch, toes, and ankle region, to retain the warming assembly in place without the need for additional fastening mechanisms. This strapless configuration can improve patient comfort, reduce setup time, and further minimize obstruction of dorsal surfaces. In certain embodiments, the warming assembly may be used as part of a modular system in which multiple assemblies are independently attachable to different extremities, enabling simultaneous warming of multiple sites while maintaining flexibility in clinical use.

Referring now to FIG. 5, an embodiment 500 of a bladder and housing is shown after being molded to the contours of a patient’s hand. The patient warming system 500 can include features described in relation to any other embodiment disclosed herein. The system 500 may include a bladder (not shown) configured to contain and circulate a heat transfer fluid, partially surrounded by a moldable housing 521. The moldable housing 521 may be configured to surround a periphery of the bladder while leaving an exposed portion of the bladder accessible, enabling direct thermal contact between the bladder and the glabrous skin of the patient’s palm. In this manner, heat transfer can be localized to target regions while avoiding coverage of dorsal vasculature.

As shown by the indentations 518 formed along the moldable housing 521, the housing may be manually shaped to conform to anatomical features of the patient’s hand, including fingers, palm, and wrist. For example, the moldable housing 521 can be wrapped over one or more fingers and/or molded around palmar surfaces of the fingers to form impressions in which the fingers can be received and supported. Similar shaping may be performed around the wrist or, in a foot application, around toes and ankle regions. Once molded, the housing retains its shape to maintain the bladder in close thermal contact with the glabrous skin while reducing relative movement between the warming assembly and the patient.

The moldable housing 521 may be configured to secure the warming assembly without the need for additional fastening mechanisms. In certain embodiments, this strapless configuration can reduce setup time, improve patient comfort, and minimize obstruction of dorsal surfaces. In other embodiments, one or more straps or attachment elements may be used in combination with the moldable housing to provide additional retention where desired.

The moldable housing 521 may include a support structure comprising a rigid, plastically deformable core 504 disposed within a cushioning layer 506. The core 504 may be formed from materials such as aluminum, malleable stainless steel, annealed copper, thermoplastic composites, or other materials capable of retaining a deformed shape after manual manipulation. The cushioning layer 506 may include foam materials such as closed-cell polyethylene foam, ethylene-vinyl acetate (EVA) foam, silicone foam, or other flexible and compressible materials configured to improve comfort and distribute pressure across the patient’s skin.

The combination of the deformable core 504 and cushioning layer 506 allows the housing 521 to be manually shaped at ambient conditions without external heating or specialized tools, enabling rapid adaptation to different patient anatomies. In some embodiments, the housing may function similarly to a moldable splint, providing both structural support and positional retention of the bladder. This configuration can improve consistency of thermal contact, enhance heat transfer efficiency, and contribute to reliable positioning during use.

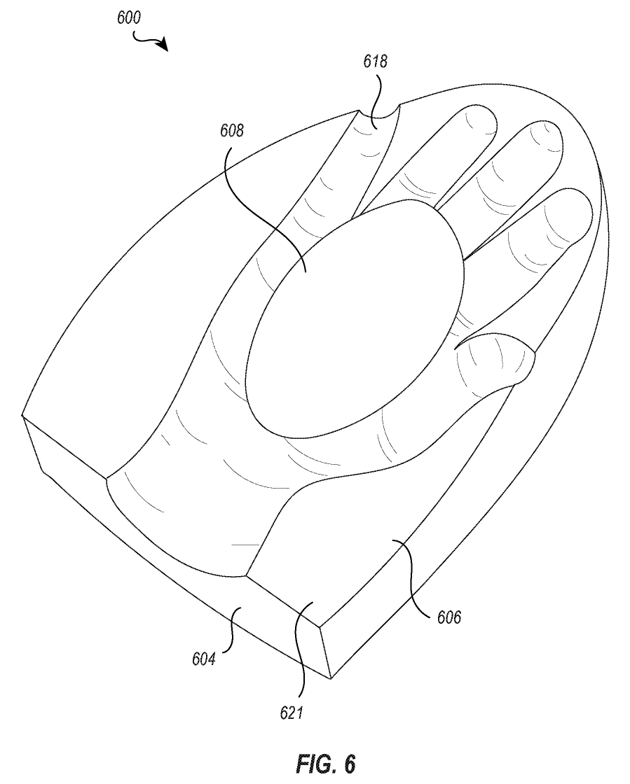

Referring now to FIG. 6, an embodiment 600 of a bladder and housing is shown, corresponding to the molded configuration previously described. The system 600 can include features described in relation to any other embodiment disclosed herein. The system 600 may include a bladder 608 configured to contain a heat transfer fluid, partially surrounded by a moldable housing 621. The moldable housing 621 can surround a periphery of the bladder while leaving an exposed portion of the bladder 608 accessible, enabling direct contact of the bladder 608 with the glabrous skin of the patient’s palm or sole. The moldable housing 621 can be utilized to support and retain the bladder 608 and to conform to other portions of the patient’s extremity.

In some embodiments, the bladder 608 may be configured to circulate the heat transfer fluid in a continuous closed-loop flow and may include internal flow paths configured to distribute the fluid across a contact surface to promote uniform heat transfer. In certain embodiments, the bladder 608 may be formed from a flexible, fluid-compatible material and may be configured as a disposable or replaceable component, optionally used in conjunction with a removable or disposable barrier layer to provide a sterile interface between the patient and reusable components of the system.

As shown by the indentations 618 visible along the moldable housing 621, the moldable housing 621 can be molded to the patient’s fingers and/or wrist (and similarly to toes and/or lower ankle for a foot application). For example, the moldable housing 621 can be wrapped over one or more fingers and/or molded around palmar surfaces of one or more fingers to create impressions in which the fingers can sit within and be secured. Similar molding (e.g., over and/or in between toes) of the housing can be utilized for foot applications. Once molded, the housing retains its shape to promote securement of the system 600 in close thermal contact with the glabrous skin surface while minimizing movement of the extremity relative to the bladder 608, and while minimizing or avoiding obstruction of dorsal vasculature.

In some embodiments, the moldable housing 621 may function as a splint-like support structure, stabilizing the extremity relative to the bladder 608 and improving consistency of thermal contact. The moldable housing 621 can in some embodiments minimize or eliminate the need for external straps or fasteners, though certain other embodiments may include both a moldable housing and one or more attachment elements.

The moldable housing 621 can include a rigid, plastically deformable core 604, which may be formed from an aluminum sheet or other suitable materials such as malleable stainless steel, annealed copper, thermoplastic composites, or other materials capable of retaining a deformed shape after manual manipulation, or combinations thereof. The core 604 can be disposed within a cushioning layer 606, which may include closed-cell polyethylene foam, ethylene-vinyl acetate (EVA) foam, silicone foam, or similar flexible, compressible materials. The combination of the rigid core 604 and the cushioning layer 606 allows the housing 621 to be manually shaped at ambient temperatures without external heating or specialized tools.

In some embodiments, the cushioning layer 606 may be configured to distribute pressure across the patient’s skin and improve comfort during use, while the core 604 provides structural retention of the molded shape. The moldable housing 621 may be configured to accommodate variations in patient anatomy, enabling a universal or adaptable fit across different hand or foot sizes. Further, the combination of conformability and structural retention may improve heat transfer efficiency by maintaining consistent contact between the bladder 610 and the glabrous skin surface. In some embodiments, the warming assembly is configured to provide a universal fit across a range of patient sizes and anatomical variations. The moldable housing may be manually shaped to conform to different hand and foot sizes, enabling a single device configuration to accommodate a wide range of patients without requiring multiple sizes. The combination of a flexible bladder and deformable housing may allow the device to maintain effective thermal contact across varying anatomical geometries while minimizing adjustment or customization by the user.

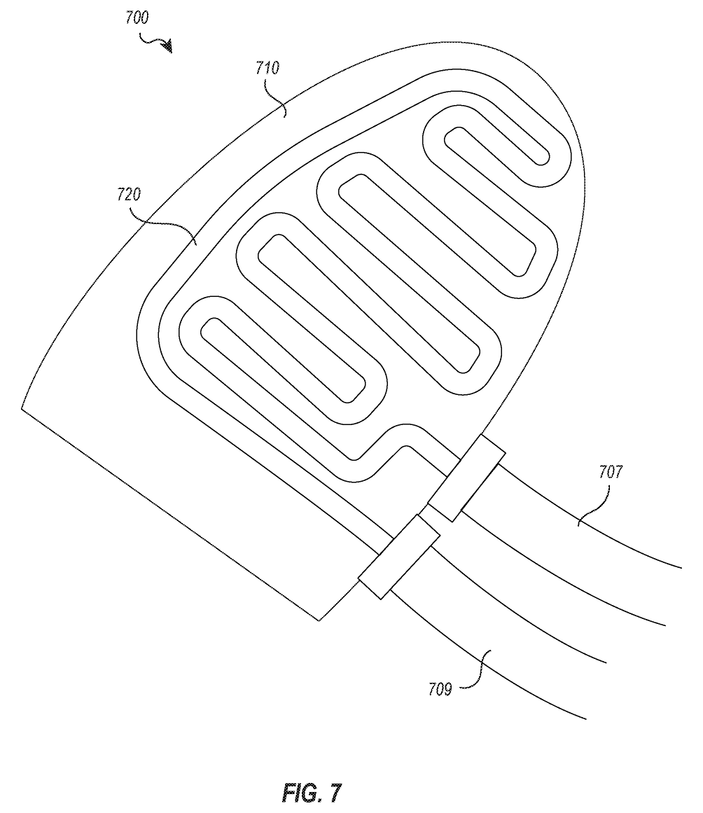

Referring now to FIG. 7, an embodiment of a bladder and fluid pathway assembly 700 is shown. Any of the other bladders disclosed herein may include such features. The system 700 includes a bladder 710 configured to contain and circulate a heat transfer fluid for direct thermal contact with a patient's glabrous skin. A heated tube 709 delivers heated heat transfer fluid from the heat exchange unit into the bladder 710. Within the bladder 710, the tube 709 is arranged in a generally spiral, coiled, or serpentine configuration, allowing the heated fluid to be distributed broadly across the contact surface area of the bladder to maximize the surface area exposed to the heated fluid, promoting efficient and uniform heat transfer throughout the bladder.

In some embodiments, the internal fluid pathway 720 may be formed as an embedded channel or conduit within the bladder 710, and may be configured to provide substantially uniform spacing across the contact surface to reduce localized temperature variations. In certain embodiments, the geometry of the serpentine or coiled pathway may be selected to balance flow resistance and heat transfer efficiency. The bladder 710 may be formed from flexible, thermally conductive materials to facilitate conformal contact with the patient while maintaining durability during repeated use or handling.

As the fluid cools after transferring heat to the patient, it is collected by a return pathway within the bladder and exits via a return tube 707. The cooled fluid is then directed back toward the heat exchange unit and pump for reheating and recirculation, maintaining a continuous closed-loop system to sustain effective warming during treatment. Continuous circulation of the heat transfer fluid through the internal pathway 720 can reduce temperature gradients and improve overall thermal transfer efficiency at the glabrous skin interface.

In some embodiments, the supply and return tubes 709, 707 may be configured to connect to upstream tubing segments via connectors, such as quick-disconnect couplings, to facilitate attachment and removal of the bladder 710 from the fluid distribution system. In certain embodiments, the bladder 710 and associated tubing may be configured as disposable components, optionally used in conjunction with a sterile barrier, while upstream components of the system remain reusable. Additionally, in some embodiments, the flow paths may be visually distinguishable, such as by color coding, to differentiate supply and return lines and reduce the likelihood of incorrect connections during setup.

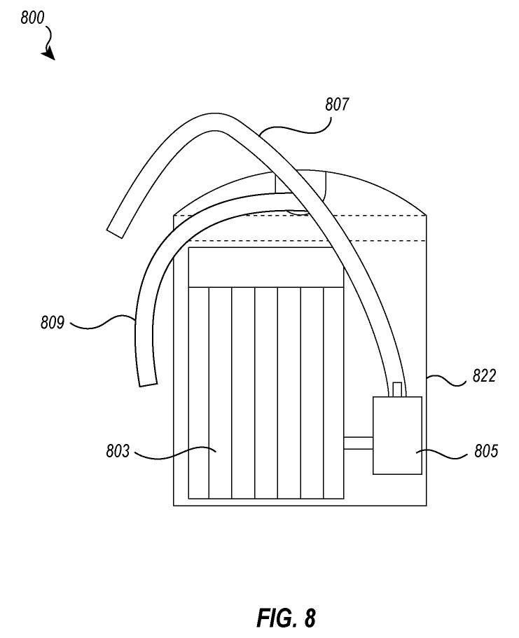

Referring now to FIG. 8, an embodiment of a patient warming system 800 is shown in a packaged configuration in which major system components are integrated within an enclosure 822. The system 800 can include features described in relation to any other embodiment disclosed herein. The enclosure 822 is configured to house at least a heat exchange assembly, fluid handling components, and associated electrical components in a compact, portable configuration suitable for use in clinical environments. In certain configurations, the system may be configured for use while a patient is seated, ambulatory, or otherwise repositioned, enabling continuous thermoregulation without interrupting treatment. In some embodiments, the system is configured to be portable and movable during use, allowing continued patient warming while the patient is not under anesthesia, such as during dialysis or other clinical procedures. The enclosure and associated components may be configured to support bedside placement, attachment to a patient support structure, or even a wearable configuration (e.g., backpack), allowing the system to remain in proximity to the patient during transport or repositioning.

The system 800 includes a heating element 803 configured to heat a heat transfer fluid. In the illustrated embodiment, the heating element 803 may be disposed within or adjacent to a reservoir/pump assembly 805. The reservoir/pump assembly 805 may be configured to contain the heat transfer fluid and to circulate the fluid through the system. In some embodiments, the heating element 803 may be disposed within the reservoir, while in other embodiments the heating element may be external to the reservoir and configured to transfer heat through a wall of the reservoir. The reservoir/pump assembly 805 may further include or be coupled to a pump configured to drive circulation of the heat transfer fluid in a closed-loop system.

A supply tube 807 is configured to deliver heated heat transfer fluid from the reservoir/pump assembly 805 to one or more downstream warming assemblies, while a return tube 809 is configured to return cooled fluid from the warming assemblies back to the reservoir/pump assembly 805 for reheating and recirculation. In some embodiments, the tubes 807, 809 may be flexible and configured for routing in a clinical setting, and may be visually distinguishable, such as by color coding, to differentiate supply and return flow paths. The system 800 may further include connectors (not shown in this view) positioned along the tubes 807, 809 to facilitate coupling and decoupling of sterile and non-sterile portions of the system, including quick-disconnect couplings configured to restrict fluid flow upon disconnection.

The enclosure 822 may comprise a rigid housing configured to protect internal components and to support integrated system packaging. In some embodiments, the enclosure 822 may be formed from materials such as plastics, composites, or metal structures, and may be configured as a portable case, toolbox-style housing, or insulated container. For example, the enclosure 822 may be configured similarly to a cooler, insulated case, or modular equipment housing, providing both structural protection and thermal insulation for the reservoir and heating components. In certain embodiments, the enclosure may include insulation layers to reduce thermal losses and improve heating efficiency of the system.

The enclosure 822 may further be configured to separate fluid-containing components from electrical components, for example by incorporating internal dividers or compartments to reduce risk of electrical exposure to moisture. In some embodiments, the enclosure 822 may include access features such as a fill port for the reservoir, removable panels for maintenance, or covers to protect fluid connections. The enclosure 822 may also include structural features configured for portability and deployment, such as handles, latches, or mounting features that allow the system to be placed on a support surface or attached to a patient support structure, such as a hospital bed.

In certain embodiments, the system 800 may be configured for portable operation, including battery-powered operation or dual-power configurations in which the system may operate using an external power source or an internal power source. The system may further include control and safety features, such as a thermostat or controller configured to regulate operation of the heating element 803 based on temperature feedback, and to automatically deactivate the heating element when a threshold temperature is exceeded. In some embodiments, temperature sensing may be performed at one or more locations within the system, such as within the reservoir, at an outlet of the heat exchange assembly, or within downstream tubing.

The integrated configuration shown in FIG. 8 enables the system 800 to function as a compact, modular, and portable patient warming unit capable of delivering heated fluid to one or more warming assemblies while maintaining separation between reusable system components and patient-contact components. This configuration can improve clinical usability, support sterile workflows, and enable deployment across a variety of treatment environments, including bedside use and patient transport.

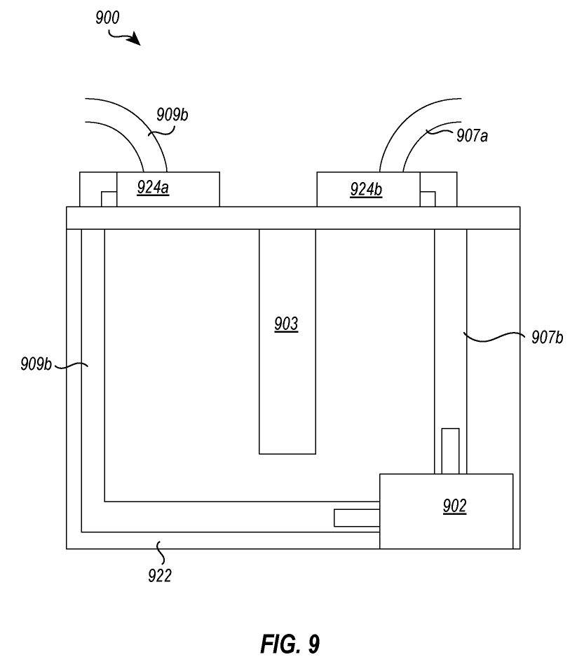

Referring now to FIG. 9, an embodiment of a patient warming system 900 is shown in an internal configuration illustrating integration of fluid distribution, heating, and circulation components within an enclosure 922. The system 900 can include features described in relation to any other embodiment disclosed herein. The enclosure 922 is configured to house system components including a heating element 903, a pump 902, fluid pathways, and distribution elements, and may provide structural support, protection, and organization of the system for use in clinical environments.

The enclosure 922 may comprise a rigid housing configured as a portable case or integrated system body. In some embodiments, the enclosure 922 may be formed as a toolbox-style housing, modular equipment case, or insulated container such as a cooler-type enclosure. The enclosure may be constructed from materials including rigid plastics, polymer composites, or metals, and may include insulation layers, such as foam or aerogel-based insulation, to reduce thermal losses and improve heating efficiency. In certain embodiments, the enclosure may define or include a fluid reservoir configured to contain the heat transfer fluid. In other embodiments, the enclosure may house a separate reservoir component positioned within the enclosure. The enclosure 922 may further include internal partitions or dividers configured to physically separate fluid-containing components from electrical components, thereby reducing risk of electrical exposure to moisture and improving system safety.

The enclosure 922 may include access and usability features, such as removable panels, hinged covers, or dedicated access ports to allow access to internal components, including manifolds, tubing connections, and the reservoir. In some embodiments, the enclosure may include a fill port accessible from an exterior surface to allow replenishment of the heat transfer fluid without disassembly. The enclosure may further include structural features to support portability and deployment, such as handles, latches, wheels, or mounting elements. In certain embodiments, the enclosure may be configured to be placed adjacent to or hung from a patient support structure, such as a hospital bed, using hooks, brackets, or straps, thereby enabling bedside use and mobility during patient transport.

The system 900 may include a heating element 903 configured to heat a heat transfer fluid within the system. In some embodiments, the heating element 903 may be disposed within a fluid-containing region, such as a reservoir defined by or contained within the enclosure 922. In other embodiments, the heating element may be external to the fluid volume and configured to transfer heat through a wall or interface, such as via a heat sheet or external heating element applied to the reservoir. This external heating configuration can reduce exposure of electrical components to fluid and improve safety. The heating element 903 may be associated with a control system including a thermostat and one or more temperature sensors, such as thermocouples, configured to measure temperature at one or more locations, including within the reservoir, at an outlet of the heating region, along supply tubing, or at or near a patient-contact interface. In some embodiments, the control system may include a feedback loop configured to regulate operation of the heating element based on sensed temperature, including automatic shutoff when a threshold temperature (e.g., approximately 110°F) is exceeded. The system may further include a display or indicator configured to provide real-time monitoring of temperature or system status.

The system 900 further includes a pump 902 configured to circulate the heat transfer fluid through the system. The pump 902 may draw cooled fluid from return pathways and direct it toward the heating element 903 for reheating, thereby establishing a continuous closed-loop circulation system. In some embodiments, the pump may comprise a peristaltic pump or other pump configured to drive fluid flow while minimizing contamination risk. The pump placement within the enclosure 922 may be selected to facilitate priming and reduce air entrapment, thereby improving flow consistency and reducing failure modes such as pressure fluctuations or bladder overexpansion.

The system 900 further includes a fluid distribution system comprising a supply-side manifold 924b and a return-side manifold 924a. The supply-side manifold 924b is configured to distribute heated heat transfer fluid to a plurality of supply flow paths, such as supply tube segments 907a, 907b, while the return-side manifold 924a collects cooled fluid from return flow paths, such as return tube segments 909a, 909b. In some embodiments, the manifold structures 924a, 924b may include a plurality of ports, such as a two-port, three-port, or four-port configuration, enabling distribution of fluid to multiple warming assemblies. In a four-port embodiment, the system may simultaneously supply heated fluid to multiple distal extremities, such as both hands and both feet, thereby increasing effective heat transfer surface area and improving warming efficiency. The manifold configuration may also allow selective use of fewer ports, for example by sealing unused ports, thereby providing a modular and scalable system architecture.

The supply and return flow paths 907a, 907b, 909a, 909b may comprise flexible tubing configured for routing within the enclosure 922 and extending outward for connection to warming assemblies. In some embodiments, the tubing may be visually distinguishable, such as by color coding (e.g., red for supply and blue for return), to reduce the likelihood of incorrect connections. The system may further include connectors disposed along the tubing, including quick-disconnect couplings configured to selectively couple and decouple components and to automatically restrict or stop fluid flow when disconnected, thereby reducing leakage and improving usability. In certain embodiments, the connectors may include keyed or gendered configurations configured to prevent misconnection of supply and return lines, thereby improving safety and reducing the risk of incorrect assembly. Additional leak-prevention features may include seals, valves, or automatic shutoff mechanisms integrated into the connectors or manifold ports.

In some embodiments, the connectors may be positioned to separate sterile and non-sterile portions of the system. For example, components within the enclosure 922, including the heating element 903 and pump 902, may be located outside a sterile field, while downstream tubing and patient-contact components may be positioned within the sterile field. This arrangement allows disposable components to be replaced between uses while maintaining reuse of the central system.

In certain embodiments, the system 900 may further include power and control subsystems integrated within the enclosure 922. These may include battery-powered operation, dual power capability (e.g., operation via both battery and external AC power), and a battery management system (BMS) configured to regulate power delivery, monitor battery condition, and improve safety. The system may include external power connectors, charging interfaces, and indicators or displays configured to provide real-time feedback regarding system operation, temperature, or power status.

The integrated configuration shown in FIG. 9 enables centralized management of heating, circulation, and fluid distribution within a compact, portable enclosure. This configuration supports efficient closed-loop circulation of the heat transfer fluid, modular connection of multiple warming assemblies, and improved clinical usability, including bedside deployment, mobility, sterile workflow compatibility, and enhanced safety through control, monitoring, and leak-prevention features.

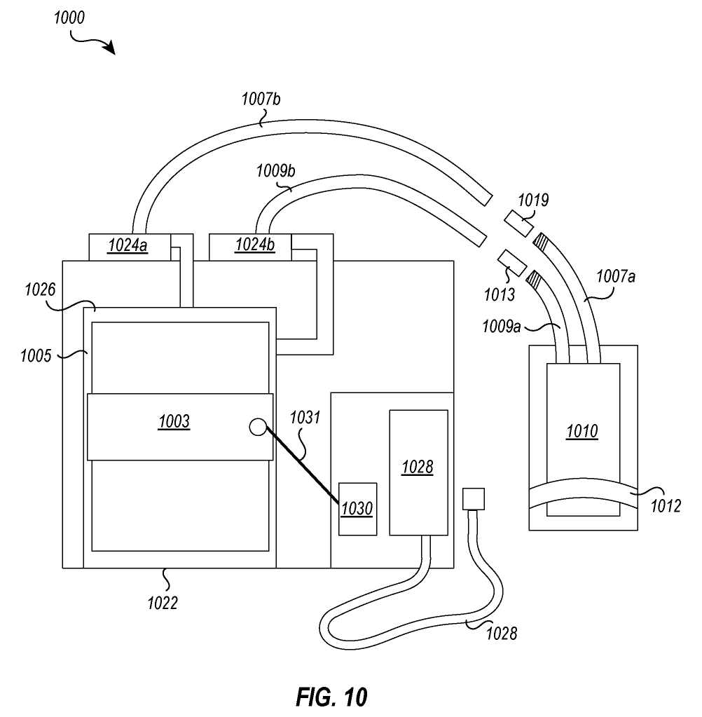

Referring now to FIG. 10, an embodiment of a patient warming system 1000 is shown in a detailed internal and functional configuration illustrating integration of heating, fluid circulation, control, and power subsystems within an enclosure 1022. The system 1000 can include features described in relation to any other embodiment disclosed herein and represents a fully integrated configuration suitable for clinical deployment.

The system 1000 may include an enclosure 1022 configured to house and protect system components, including a heating element 1003, a reservoir 1026, insulation 1005, fluid pathways, connectors, and control and power subsystems. The enclosure 1022 may comprise a rigid housing formed from materials such as plastics, polymer composites, or metals, and may be configured as a portable case, toolbox-style housing, modular equipment enclosure, or insulated container such as a cooler-type structure. In certain embodiments, the enclosure 1022 may include internal partitions configured to separate fluid-containing components from electrical components to reduce risk of electrical exposure to moisture and improve safety. The enclosure 1022 may further include structural features such as handles, latches, mounting hooks, or brackets configured to facilitate portability and placement adjacent to or attachment to a patient support structure, such as a hospital bed, thereby enabling bedside use and patient transport.

The system 1000 includes a reservoir 1026 configured to contain a heat transfer fluid, such as water, saline, or a glycol solution. The reservoir 1026 may be disposed within the enclosure 1022 and may be configured as a removable or refillable component. In some embodiments, the enclosure 1022 itself may define or include the reservoir volume, while in other embodiments the reservoir 1026 may be a discrete container positioned within the enclosure. The reservoir 1026 may be thermally coupled to the heating element 1003 and may be surrounded by insulation 1005 configured to reduce thermal losses and improve heating efficiency. The insulation 1005 may include materials such as foam, aerogel-based insulation, or other thermally resistive materials.

The heating element 1003 is configured to heat the heat transfer fluid contained within the reservoir 1026. In some embodiments, the heating element 1003 may be disposed within the reservoir, while in other embodiments the heating element may be external to the reservoir and configured to transfer heat through a wall of the reservoir, such as via a heat sheet or external heating element. This external heating configuration can reduce exposure of electrical components to fluid and improve system safety. The heating element 1003 may be controlled by a thermostat 1030 configured to regulate operation of the heating element based on temperature feedback.

The system 1000 further includes a thermocouple 1031 configured to measure temperature of the heat transfer fluid and provide feedback to the thermostat 1030. The thermocouple 1031 may be positioned at one or more locations within the system, such as within the reservoir 1026, at an outlet of the heating region, along a supply flow path, or at or near a patient-contact interface. In some embodiments, the thermostat 1030 and thermocouple 1031 may form part of a closed-loop control system configured to maintain the heat transfer fluid within a desired temperature range, such as approximately 95°F to 120°F, and to automatically deactivate the heating element 1003 when a threshold temperature is exceeded (e.g., approximately 110°F). The thermostat 1030 may further include a display or user interface to provide real-time monitoring of temperature and system status.

The system 1000 may include a fluid distribution system comprising supply and return flow paths. Supply flow paths, such as tubing segments 1007a and 1007b, are configured to deliver heated heat transfer fluid from the reservoir 1026 to one or more downstream warming assemblies. Return flow paths, such as tubing segments 1009a and 1009b, are configured to return cooled fluid from the warming assemblies back to the reservoir 1026 for reheating. In the illustrated embodiment, tubing segments 1007a, 1009a may correspond to sterile portions of the system, while tubing segments 1007b, 1009b may correspond to non-sterile portions of the system. This arrangement enables separation between sterile and non-sterile components, allowing reusable components within the enclosure 1022 to remain outside a sterile field while disposable patient-contact components are used within the sterile field.

The system 1000 may further include connectors 1013 and 1019 disposed along the supply and return flow paths. The connectors 1013, 1019 may comprise quick-disconnect couplings configured to selectively couple and decouple sterile and non-sterile tubing segments. In some embodiments, the connectors may be configured to automatically restrict or stop fluid flow when disconnected, thereby preventing leakage and improving safety. The connectors may further include keyed or gendered configurations configured to prevent misconnection of supply and return lines. Additional leak-prevention features may include internal valves, seals, or shutoff mechanisms integrated into the connectors.

In some embodiments, the fluid distribution system may include a manifold 1024a configured to distribute heated fluid to multiple supply flow paths and another manifold 1024b to collect return flow from multiple return paths. The manifold may include a multi-port configuration, such as a four-port architecture, allowing simultaneous connection of multiple warming assemblies (e.g., two hands and two feet). This manifold-based distribution enables modular and scalable system operation.

The system 1000 further includes a warming assembly comprising a bladder 1010 configured to receive heated heat transfer fluid and to contact glabrous skin of a patient’s distal extremity. The bladder 1010 may include internal flow pathways configured to distribute the fluid across a contact surface to promote uniform heat transfer. The bladder 1010 may be formed from flexible, fluid-compatible materials and may be configured as a disposable component. In some embodiments, the bladder 1010 may be used in conjunction with a disposable barrier layer to provide a sterile interface.

The warming assembly may further include a strap 1012 configured to secure the bladder 1010 to the patient’s extremity. The strap 1012 may be positioned across a dorsal surface of the extremity to maintain contact between the bladder and glabrous skin while leaving dorsal vasculature accessible for clinical procedures. The strap 1012 may be adjustable and may include fastening mechanisms such as hook-and-loop fasteners, buckles, snaps, or similar devices. In some embodiments, the strap may be omitted and a moldable housing (not shown in this figure) may be used to secure the bladder.

The system 1000 further includes a power line 1028 configured to supply electrical power to the heating element 1003, pump, and associated control components. In some embodiments, the system may be configured for operation using an external power source, while in other embodiments the system may include an internal power source, such as a battery. In certain embodiments, the system may support dual power operation, including both battery and external AC power. The system may further include a battery management system (not shown) configured to regulate power delivery, monitor battery condition, and improve safety and reliability.

In operation, the heating element 1003 may heat the heat transfer fluid within the reservoir 1026, and the fluid can be circulated through the supply flow paths 1007a, 1007b to the bladder 1010. Heat is transferred from the fluid to the patient through the glabrous skin interface, and the cooled fluid is returned through the return flow paths 1009a, 1009b to the reservoir 1026 for reheating. The closed-loop configuration, combined with controlled heating and modular distribution, may enable efficient and consistent patient warming while supporting sterile workflow, portability, and safe operation. In some embodiments, the system includes a control system comprising a temperature sensor and a controller configured to regulate operation of the heating element based on temperature of the heat transfer fluid.

### Example Aspects

The following clauses represent a non-exhaustive list of example features that may be included in the disclosed patient warming system.

Clause 1. A patient warming system comprising: a heat exchange unit comprising a heat transfer fluid and a heating element configured to heat the heat transfer fluid; a pump configured to circulate the heat transfer fluid; a fluid distribution system fluidically coupled to the heat exchange unit, the fluid distribution system comprising a supply flow path and a return flow path; and a warming assembly comprising (i) a bladder configured to receive the heat transfer fluid, and (ii) a moldable housing partially surrounding the bladder and configured to conform to a distal extremity of a patient while leaving dorsal vasculature exposed.

Clause 2. The system of clause 1, wherein the fluid distribution system comprises a manifold having an inlet and a plurality of outlet ports, each outlet port being fluidically coupled to a respective warming assembly.

Clause 3. The system of any one of the preceding clauses, further comprising a connector configured to selectively couple and decouple portions of the fluid distribution system.

Clause 4. The system of clause 3, wherein the connector comprises a quick-disconnect coupling configured to restrict fluid flow when disconnected, wherein the connector is positioned between a sterile portion and a non-sterile portion of the system.

Clause 5. The system of any one of the preceding clauses, wherein the moldable housing comprises a support material comprising a plastically deformable core and/or a cushioning layer.

Clause 6. The system of any one of the preceding clauses, wherein the heat exchange unit is configured to maintain the heat transfer fluid within a temperature range of approximately 95°F to 120°F.

Clause 7. The system of any one of the preceding clauses, further comprising a temperature sensor configured to measure a temperature of the heat transfer fluid and optionally a controller configured to regulate operation of the heating element based on the measured temperature, wherein the controller is configured to deactivate the heating element when a threshold temperature is exceeded.

Clause 8. The system of any one of the preceding clauses, wherein the heating element is disposed within a reservoir of the heat exchange unit.

Clause 9. The system of any one of the preceding clauses, wherein the heat transfer fluid comprises one or more of water, saline, or a glycol.

Clause 10. The system of any one of the preceding clauses, wherein the system comprises a plurality of warming assemblies, optionally wherein each warming assembly is coupled to the fluid distribution system via a respective supply flow path and return flow path.

Clause 11. The system of any one of the preceding clauses, wherein the warming assembly further comprises an attachment element configured to secure the warming assembly to the distal extremity, such as wherein the warming assembly comprises one or more straps, the straps optionally positioned so as to pass over the wrist and/or knuckles of the patient’s hand when worn by the patient to leave the dorsum exposed between the knuckles and the wrist.

Clause 12. The system of any one of the preceding clauses, further comprising an enclosure housing at least the heat exchange unit and the pump.

Clause 13. A patient warming system comprising: a heat exchange unit configured to heat a heat transfer fluid; a pump configured to circulate the heat transfer fluid; a manifold fluidically coupled to the heat exchange unit, the manifold comprising a plurality of outlet ports; a plurality of warming assemblies each fluidically coupled to a respective outlet port of the manifold, each warming assembly comprising a bladder configured to contact glabrous skin of a distal extremity; and a plurality of return flow paths configured to return fluid from the warming assemblies to the heat exchange unit; and a control system configured to regulate heating of the heat transfer fluid.

Clause 14. The system of clause 13, wherein the control system comprises a temperature sensor and a controller, the controller being configured to regulate operation of the heat exchange unit based on a measured temperature of the heat transfer fluid.

Clause 15. The system of clause 14, wherein the temperature sensor comprises a thermocouple configured to measure temperature of the heat transfer fluid at or downstream of the heat exchange unit.

Clause 16. The system of any one of clauses 13-15, further comprising an enclosure housing at least the heat exchange unit and the pump.

Clause 17. The system of clause 16, wherein the enclosure is configured to separate fluid-containing components from electrical components.

Clause 18. The system of any one of clauses 13-17, wherein a connector comprises a quick-disconnect coupling configured to restrict fluid flow when disconnected and to separate a sterile portion of the system from a non-sterile portion of the system.

Clause 19. The system of any one of clauses 13-18, wherein: the system comprises a plurality of warming assemblies configured for simultaneous warming of a plurality of distal extremities; and each warming assembly further comprises an attachment element configured to secure the warming assembly to the distal extremity, the attachment element comprising a strap and/or a moldable housing configured to secure the warming assembly to the distal extremity.

Clause 20. A patient warming system comprising: a heat exchange unit comprising a heat transfer fluid and a heating element configured to heat the heat transfer fluid; a pump configured to circulate the heat transfer fluid; a fluid distribution system fluidically coupled to the heat exchange unit, the fluid distribution system comprising a manifold having an inlet and a plurality of outlet ports; a plurality of warming assemblies each fluidically coupled to a respective outlet port of the manifold, each warming assembly comprising: a bladder configured to receive the heat transfer fluid and to conform to glabrous skin of a distal extremity of a patient while leaving dorsal vasculature of the distal extremity uncovered; a moldable housing at least partially surrounding the bladder and configured to conform to the distal extremity; and an attachment element configured to secure the warming assembly to the distal extremity; a plurality of supply flow paths extending from the manifold to the plurality of warming assemblies and configured to deliver heated heat transfer fluid to the plurality of warming assemblies; and a plurality of return flow paths extending from the plurality of warming assemblies to the heat exchange unit and configured to return cooled heat transfer fluid.

### Additional Terms & Definitions

Where proportions, dimensions, or relative positioning are disclosed herein in reference to an anatomical feature (e.g., a hand or foot), it can be understood in terms of the anatomical feature with size according to the average U.S. adult. Notwithstanding, adaptations can be made for various patient sizes or clinical contexts.

As used herein, the term “moldable” when referring to the housing structure indicates that the material can be manually shaped at ambient environmental conditions expected in a caretaker setting (e.g., operating room), without requiring external heat or specialized tools, and that once shaped, the material retains the molded configuration until intentionally manipulated to another shape.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element or component of a described embodiment, any of the possible alternatives listed for that element or component may generally be used individually or in combination with one another, unless implicitly or explicitly stated otherwise.

When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “bladder”) may also include two or more such referents.

The embodiments disclosed herein should be understood as comprising/including disclosed components, and may therefore include additional components not specifically described. Optionally, the embodiments disclosed herein can be understood to “consist of” or “consist essentially of” any combination of disclosed components. Some embodiments are free of components that are not specifically described. That is, non-disclosed components may optionally be completely omitted or essentially omitted from the disclosed embodiments. For example, heating mechanisms, patient attachment mechanisms, and/or patient heating contacts/interfaces not disclosed herein may optionally be omitted.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A patient warming system comprising:
a heat exchange unit comprising a heat transfer fluid and a heating element configured to heat the heat transfer fluid;
a pump configured to circulate the heat transfer fluid;
a fluid distribution system fluidically coupled to the heat exchange unit, the fluid distribution system comprising a supply flow path and a return flow path; and
a warming assembly comprising:
a bladder configured to receive the heat transfer fluid, and
a moldable housing partially surrounding the bladder and configured to conform to a distal extremity of a patient while leaving dorsal vasculature exposed.

2. The system of claim 1, wherein the fluid distribution system comprises a manifold having an inlet and a plurality of outlet ports, each outlet port being fluidically coupled to a respective warming assembly.

3. The system of claim 1, further comprising a connector configured to selectively couple and decouple portions of the fluid distribution system.

4. The system of claim 3, wherein the connector comprises a quick-disconnect coupling configured to restrict fluid flow when disconnected, wherein the connector is positioned between a sterile portion and a non-sterile portion of the system.

5. The system of claim 1, wherein the moldable housing comprises a support material comprising a plastically deformable core and a cushioning layer.

6. The system of claim 1, wherein the heat exchange unit is configured to maintain the heat transfer fluid within a temperature range of approximately 95°F to 120°F.

7. The system of claim 1, further comprising a temperature sensor configured to measure a temperature of the heat transfer fluid and a controller configured to regulate operation of the heating element based on the measured temperature, wherein the controller is configured to deactivate the heating element when a threshold temperature is exceeded.

8. The system of claim 1, wherein the heat transfer fluid comprises one or more of water, saline, or a glycol.

9. The system of claim 1, wherein the system comprises a plurality of warming assemblies, wherein each warming assembly is coupled to the fluid distribution system via a respective supply flow path and return flow path.

10. The system of claim 1, wherein the warming assembly further comprises an attachment element configured to secure the warming assembly to the distal extremity.

11. The system of claim 10, wherein the attachment element comprises one or more straps configured to pass over a dorsal side of the hand leaving exposed the dorsal surface between the knuckles and wrist.

12. The system of claim 1, further comprising an enclosure housing at least the heat exchange unit and the pump.

13. A patient warming system comprising:
a heat exchange unit configured to heat a heat transfer fluid;
a pump configured to circulate the heat transfer fluid;
a manifold fluidically coupled to the heat exchange unit, the manifold comprising a plurality of outlet ports;
a plurality of warming assemblies each fluidically coupled to a respective outlet port of the manifold, each warming assembly comprising a bladder configured to contact glabrous skin of a distal extremity; and
a plurality of return flow paths configured to return fluid from the warming assemblies to the heat exchange unit; and
a control system configured to regulate heating of the heat transfer fluid.

14. The system of claim 13, wherein the control system comprises a temperature sensor and a controller, the controller being configured to regulate operation of the heat exchange unit based on a measured temperature of the heat transfer fluid.

15. The system of claim 14, wherein the temperature sensor comprises a thermocouple configured to measure temperature of the heat transfer fluid at or downstream of the heat exchange unit.

16. The system of claim 13, further comprising an enclosure housing at least the heat exchange unit and the pump.

17. The system of claim 16, wherein the enclosure is configured to separate fluid-containing components from electrical components.

18. The system of claim 13, wherein a connector comprises a quick-disconnect coupling configured to restrict fluid flow when disconnected and to separate a sterile portion of the system from a non-sterile portion of the system.

19. The system of claim 13, wherein:
the system comprises a plurality of warming assemblies configured for simultaneous warming of a plurality of distal extremities; and
each warming assembly further comprises an attachment element configured to secure the warming assembly to the distal extremity, the attachment element comprising a strap and/or a moldable housing configured to secure the warming assembly to the distal extremity.

20. A patient warming system comprising:
a heat exchange unit comprising a heat transfer fluid and a heating element configured to heat the heat transfer fluid;
a pump configured to circulate the heat transfer fluid;
a fluid distribution system fluidically coupled to the heat exchange unit, the fluid distribution system comprising a manifold having an inlet and a plurality of outlet ports;
a plurality of warming assemblies each fluidically coupled to a respective outlet port of the manifold, each warming assembly comprising:
a bladder configured to receive the heat transfer fluid and to conform to glabrous skin of a distal extremity of a patient while leaving dorsal vasculature of the distal extremity uncovered;
a moldable housing at least partially surrounding the bladder and configured to conform to the distal extremity; and
an attachment element configured to secure the warming assembly to the distal extremity;
a plurality of supply flow paths extending from the manifold to the plurality of warming assemblies and configured to deliver heated heat transfer fluid to the plurality of warming assemblies; and
a plurality of return flow paths extending from the plurality of warming assemblies to the heat exchange unit and configured to return cooled heat transfer fluid.
